(19) United States
(12) Patent Application Publication
(10) Pub. No.: US 2002/0038066 A1
(43) Pub. Date: Mar. 28, 2002

(54) FIXED CATALYTIC BED REACTOR

(76) Inventors: VINCENT A. STRANGIO, WEST ORANGE, NJ (US); FRITS M. DAUTZENBERG, MAHWAH, NJ (US); HANS PETER ALEXANDER CALIS, NOOTDORP (NL); AVINASH GUPTA, BLOOMFIELD, NJ (US)

Correspondence Address: CARELLA BYRNE BAIN GILFILLAN, CECCHI STEWART & OLSTEIN, 6 BECKER FARM ROAD, ROSELAND, NJ, 07068

(21) Appl. No.: 09/273,848

(22) Filed: Mar. 22, 1999

Related U.S. Application Data
(60) Provisional application No. 60/078,996, filed on Mar. 23, 1998.

(51) Int. Cl. C07C 4/02

(52) U.S. Cl. 585/440; 422/196; 422/197; 422/193; 585/920

(57) ABSTRACT

A fixed particle bed in a vessel wherein the bed is a structured bed in a plurality of flow channels with the cross-section of the bed in each channel being from 1 to 20 particles, more preferably 1 to 10 particles.

This application claims the priority of U.S. Provisional Application 60/078,996 filed on Mar. 23, 1998.

This invention relates to a packed particle bed and in particular to a packed or fixed catalyst bed, vessels or reactors containing such beds and the use thereof.

There are many applications for a packed particulate bed. For example, such packed beds have been used in absorbers, as a packed or fixed bed of catalyst for catalytic reactors, etc.

For example, fixed beds of catalysts are used in many chemical processes in a variety of reactor types. The chemical reactions may be exothermic or endothermic. The reactors themselves may be trickle bed reactors and they may contain several beds with interstage heating or cooling. The reactors may be radial type reactors where a low pressure drop is desirable. The latter type reactor is generally used where low pressure drop is required, such as, for example, in the manufacture of styrene.

In one aspect, the present invention is directed to improvements in packed particulate beds which are in a vessel or tube wherein at least a portion of the vessel includes at least one framework which divides the vessel into a plurality of flow channels with adjacent flow channels having at least one common or contiguous wall. At least a portion of the flow channels includes a bed of particles wherein the cross-section of the flow channel and the size of the particles is such that in a cross-sectional plane there is at least 1 and no greater than 20 units of particle. In a preferred embodiment, the flow channels are parallel to each other.

Thus, in accordance with this aspect of the present invention, the particle bed is a structured bed of particles rather than a random bed of particles.

Although the cross-section of the flow channel may contain up to 20 units of particles (each particle is one unit), in general, the number of units does not exceed 15 and more generally does not exceed 10. In preferred embodiments, the number of particle units in the cross-sectional area does not exceed 4.

In accordance with an aspect of the invention, the particles are non-randomly packed in the bed with the size of the particles and the amount of particles in the non-randomly packed bed being selected to provide a desired pressure drop and void volume for the bed. In one embodiment the pressure drop is less than that which results from using the same weight of particles in a randomly packed bed.

In a preferred embodiment, the particles are catalytic particles and the bed is a fixed bed of catalyst in a reaction vessel which includes a framework which provides for a plurality of flow passages at least a portion of which and in most cases all of which contain a structured bed of catalyst in accordance with the invention.

In accordance with an embodiment of the invention, the structured catalyst bed may be formed by providing the reactor with a framework which in effect divides the reactor into a plurality of elongated cells or chambers within the reactor, at least a portion of which confines and contains particles therein, with particles being stacked within the elongated cells or chambers.

The dimensions of each of the cells or chambers which define flow passages are coordinated with the size of the particles such that on a plane perpendicular to the direction of flow, there is from 1 to 20 particle units with such particles being stacked upon each other to form a bed in each chamber having a width or cross-section of from 1 to 20 particles, or as hereinabove indicated from 1 to 15 or from 1-10 or from 1-4 particle units.

In a single vessel, the size of the cells or chambers may be the same or different. In addition, the size of the particles may vary from chamber to chamber or may be of the same size in each chamber, provided that the particles are in the form of a structured bed. In fact, the particle size may vary within a chamber or flow passage.

The framework may form cells or chambers of different sizes and shapes and it is within the scope of the invention that a single vessel may contain cells of different sizes and shapes.

Similarly, the particles packed in a single cell or in different cells may be different from each other in size or shape and/or be the same. Similarly, the particles may be different in function from cell to cell or within a cell, e.g., different catalysts or they may be the same.

The framework may be comprised of a single framework or multiple frameworks, each of which divides at least a portion of the vessel into a plurality of cells or flow channels. When multiple frameworks are used, they may be the same or of different sizes and shapes. In addition, they may be positioned, arranged or stacked in different ways to provide different flow patterns.

For example, the frameworks may be arranged adjacent to each other with or without stacking of framework in the direction of flow.

The fluid which flows through the packed beds may or may not be reactants and may be a gas and/or liquid and/or multiple gases and/or liquids.

The vessel which contains the framework and bed may be of a variety of sizes and shapes including but not limited to tubular reactors, spherical reactors, etc. In each case, the vessel or tube includes a framework which divides at least a portion of the vessel or tube into a plurality of cells or chambers which define flow passages, with at least a portion of such passages including a structured bed of particles in accordance with the invention.

As hereinabove indicated all or a portion of the cells may include the structured bed of particles. When less than all of the cells include a bed of particles, in general, from 10 to 50% of the cross-sectional area of the framework is comprised of cells which do not contain particles.

The framework confines the particles within the cells or chambers and in the case where the framework is porous (including holes), the size of the pores or holes is less than the particles within the cell or chamber to confine the particles therein.

In a preferred embodiment, the structured bed of particles has a flow tortuosity therethrough of about 1; i.e., there is at least one unimpeded flow path through the bed, (a straight flow path uninterrupted by particles).

Framework which divides the vessel into a plurality of cells or chambers may be any one of a wide variety of structures including but not limited to high porosity structures such as monoliths. Such monolith structures may be fabricated from a variety of materials, with ceramics or metals or combinations thereof being generally preferred. The monolith structure may be comprised of different cell sizes or shapes including but not limited to square cells, rectangular, polygonal, ellipsoidal, triangular, sinusoidal or hexagonal cells, or cells with internal fins or ribs, etc. or may for example be arranged in spirals. In addition, the monolith structure can be formed in a variety of sizes to provide a wide variety of number of cells per structure. For example, such monolith structure may be comprised, for example, of 16 cells per square inch up to about 400 cells per square inch. The monolith structure is provided with a support screen at the bottom in order to contain or support the particles.

The framework may be porous or non-porous. For example, the framework which defines the cells may be made of a wire mesh or screen for example woven or sintered or may be formed from a porous or non-porous , metal, plastic, glass, ceramic or composite, etc.

Similarly, the framework may also include a catalyst, for example, a catalyst coated on or embedded in the framework structure, which catalyst may be the same as or be different from the catalyst which is in the form of a structured bed within the cells formed by the framework.

The present invention further relates to a catalyst framework and a structured catalyst bed therein which may be used in a catalytic reactor. In accordance with this aspect, the framework may form one or more cells or chambers which have a structured catalyst bed therein wherein the size of the catalyst units used for the bed are coordinated with the dimensions of the cell such that the bed cross-section is comprised of a number of catalyst units, as hereinabove described.

The height of the monolith structure and the height of the catalyst bed which is non-randomly packed in each of the cells or chambers is dependent on the desired height of the catalyst bed for a particular reaction. The selection of a suitable height is deemed to be within the skill of those in the art from the teachings herein.

The reactor may include several monolith structures stacked on top of each other, and they may be stacked in a manner such as to provide for interstage heating or quenching or separation (distillation) of the fluids and/or staged addition of reactants within the reactor.

The catalyst, as well as the dimensions of the chamber and catalyst may be tailored to the desired process. In cases where the mass transfer resistances are high, one would use small catalyst particles in smaller cells so as to maximize the surface area for mass transfer, with such small catalyst particles being formed in a non-randomly packed bed. If the reaction is slow and controlled by kinetics, one would want to maximize the mass of catalyst per unit volume.

The number of catalyst elements which are packed into each bed or in a preferred embodiment into each cell or chamber of a monolith or framework will also be selected depending upon the desired pressure drop and desired void fraction. The number of catalyst elements, determined on a horizontal plane with respect to the chamber or bed, affects the void volume, with the void volume decreasing as the number of catalyst elements increases.

The catalyst particles employed in the fixed bed may be in a wide variety of forms including but not limited to extrudates, beads, spheres, cylinders, rings, ribbed, etc. The selection of a particular type of catalyst is deemed to be within the scope of those skilled in the art from the teachings herein.

Similarly, the selection of a particular framework for dividing the reactor into a plurality of cells or chambers, is also deemed to be within the scope of those skilled in the art from the teachings herein.

Similarly, the selection of a particular catalyst is dependent upon the particular reaction to be effected in the fixed bed catalytic reactor. The selection of an appropriate catalyst is deemed to be within the scope of those skilled in the art from the teachings herein.

The present invention is applicable to a wide variety of catalytic reactions in a fixed catalyst bed. The present invention is particularly applicable to those reactions where a low bed pressure drop is desirable or necessary or where small particles are required to enhance mass transfer. Thus, for example, the use of a fixed bed catalytic reactor in accordance with the present invention may be used for catalytic cracking to produce ethylene or propylene or the production of styrene from ethylbenzene, or dehydrogenation to produce unsaturates, e.g., propane to propylene, or butane to butylene or butane to iso-butylene.

The invention will be further described with respect to the accompanying drawings, wherein:

The drawings are schematic representations of structured catalyst beds in accordance with the invention.

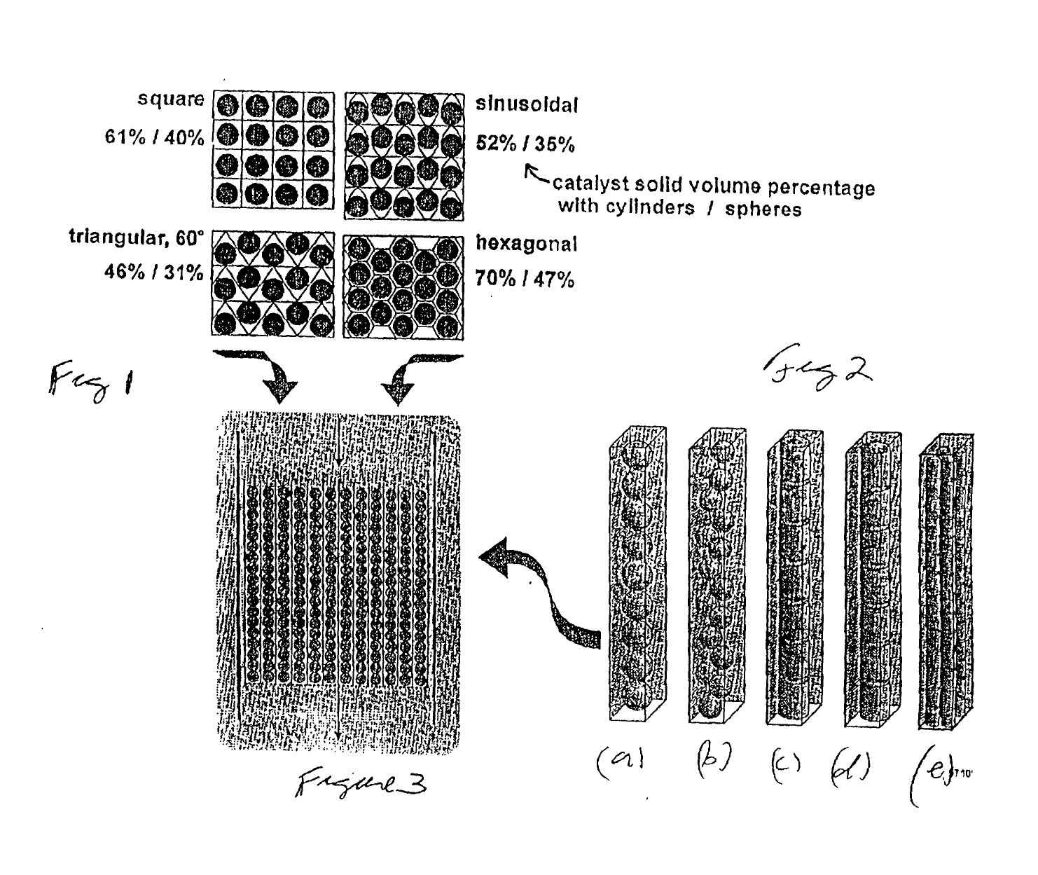

As shown in FIG. 1 of the drawings, the framework may be designed to provide cells of a variety of shapes, such as sequence, sinusoidal, triangular and hexagonal. As shown in FIG. 1, each cell contains a single catalyst unit or element which, for example, may be in the form of a cylinder, bead, etc. Although a single catalyst unit (in cross-section) is shown in each cell, as hereinabove indicated, more than one catalyst unit may be used (in cross-section) in each cell.

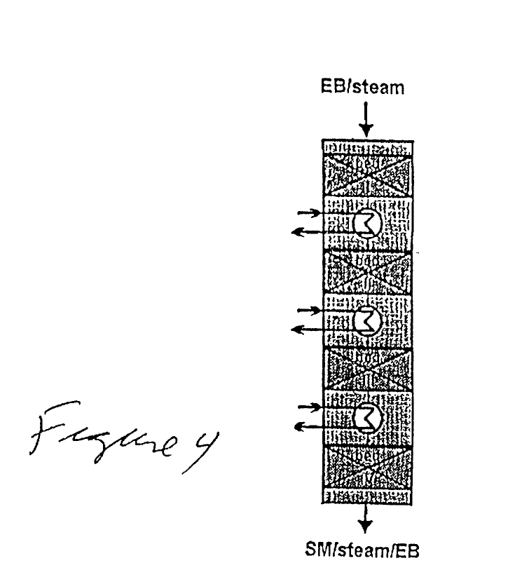

FIG. 2 of the drawings illustrates examples of single catalyst cells in which the structured catalyst bed (FIG. 2a) is comprised a single catalyst unit (in cross-section) in the form of a bead with the catalyst units being stacked to form a structured bed in alignment with each other. In FIG. 2b, the cell contains a single unit in cross-section, however, the cell dimension is such that the catalyst units (in the form of a sphere) are offset from each other in the direction of flow.

FIGS. 2B and 2C show stacked catalyst cylinders in a cell in which the cell cross-section includes a single unit.

FIG. 2D shows a cell in which the structured bed is comprised, in cross-section, of four catalyst units, in the form of stacked aligned catalyst cylinders or extrudates.

FIG. 3 illustrates a reactor which contains a fixed catalyst bed comprised of a framework forming a plurality of cells each of which includes a structured catalyst bed comprised of a single catalyst unit in cross-section.

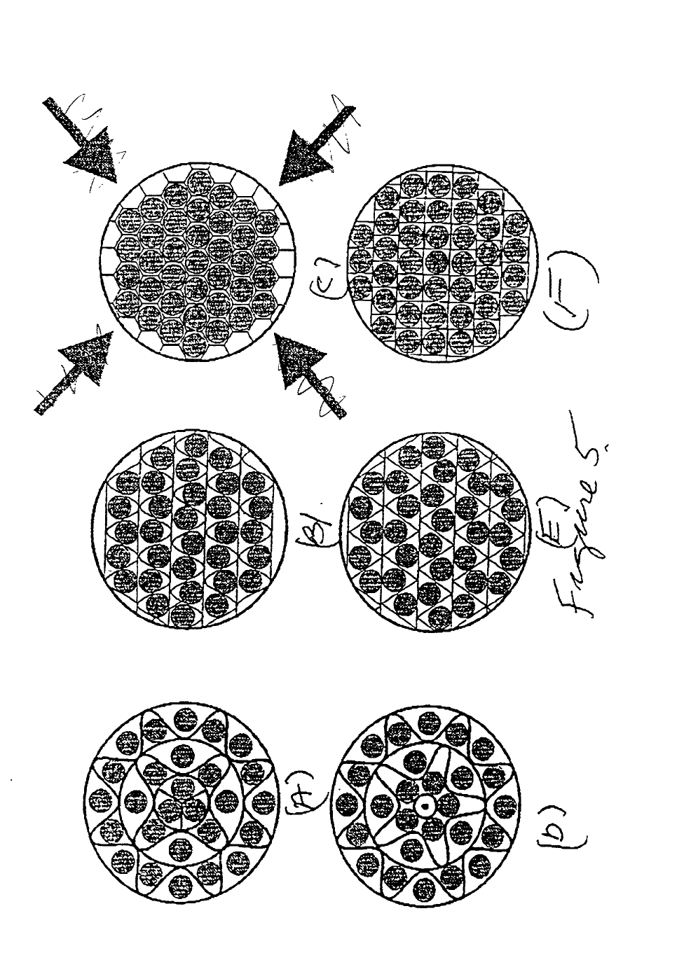

FIG. 4 is a schematic representation of a reactor for producing styrene from ethylbenzene in which each of the four catalyst beds is a structured catalyst bed in accordance with the invention.

The reactor is operated at an inlet pressure of about 9 psig and each of the structured catalyst beds is designed to provide a pressure drop of about 3 psig through the reactor.

The inlet temperature to each bed is about 600°-640° C. and the interbed heating provides for heating effluent from each bed which is at a temperature of about 530°-580° C. to an inlet temperature for the subsequent bed of about 600°-640° C.

The space velocity for the reactor is about 1.0 to 1.3 and conversion to styrene is about 65% to 75%. The steamto-feed ratio is about 1.0.

In prior art processes, in order to achieve a conversion of about 65%, with a pressure drop of about 3 psig, two reactors with random catalytic beds are required, with the space velocity in the first reactor being about 1.0 and, in the second reactor, about 1.0 to achieve an overall space velocity of about 0.5. In addition, the steam to ethylbenzene ratio is about 1.5.

Thus, by using a structured bed in accordance with the invention, catalyst can be reduced by about 50%, with lower steam requirements and only one reactor shell is required.

FIG. 5 shows a simplified schematic representation of reactor cross-sections which incorporate structured catalyst beds of the present invention in which the framework defining the cells have different shapes. In FIGS. 5A, B, C, E and F, each of the cells, in cross-section, includes a single catalyst unit. In FIG. 5D, the reactor contains a central cell which, in cross-section, contains four catalyst units with each of the remaining cells containing a single catalyst unit, in cross-section.

Although FIG. 4 described a reactor for styrene production, the present invention may be used for a wide variety of reactors for a wide variety of reactions. Thus, for example, as representative examples of other reactions, there may be mentioned: ethylene oxide production, olefin disproportionation or metathesis, formaldehyde production, acrolein production, DME production, methanol production, catalytic reforming, maleic anhydride production, selective hydrogenation processes, alkane dehydrogenation (e.g., propane to propylene), catalytic distillation reactions, hydrodesulphurization or other hydrotreating, aromatic alkylation reaction processes, phthalic anhydride production, bisphenol A production, acrylic acid production, acrylonitrile production, VOC abatement processes, NO abatement processes, absorption processes, and linear alkylbenzene formation.

Numerous modifications and variations of the present invention are possible in light of the above teachings and, therefore, within the scope of the appended claims, the invention may be practiced otherwise than as particularly described.

What is claimed is:

1. An apparatus comprising:
a vessel, at least one framework in at least a portion of the vessel forming a plurality of flow channels, with adjacent flow channels having at least one common wall, a bed of particles in at least a portion of the flow channels, wherein a bed in a channel has a cross-section of at least one and no more than twenty particles.

2. The apparatus of claim 1 wherein the particles are confined in the flow channels.

3. The apparatus of claim 2 wherein the particles are catalyst particles.

4. The apparatus of claim 3 wherein the bed cross-section in a channel is from 1-10 catalyst units.

5. The apparatus of claim 4 wherein the bed cross-section is 1-4 catalyst units.

6. The apparatus of claim 3 wherein the flow channels that include particles include at least one flow path uninterrupted by particles.

7. The apparatus of claim 3 wherein the framework is porous.

8. The apparatus of claim 3 wherein the framework is non-porous.

9. The apparatus of claim 3 wherein the particles are non-randomly packed in the flow channels.

10. The apparatus of claim 9 wherein only a portion of the flow channels include particles.

11. The apparatus of claim 9 wherein all of the flow channels include particles.

12. A process for producing a product by a chemical reaction, comprising:
effecting the chemical reaction in the apparatus of claim 3.

13. The process of claim 12 wherein the chemical reaction converts ethylbenzene to styrene.

14. A process for producing a product by a chemical reaction, comprising:
effecting the chemical reaction in the apparatus of claim 4.

15. A process for producing a product by a chemical reaction, comprising:
effecting the chemical reaction in the apparatus of claim 5.

16. A process for producing a product by a chemical reaction, comprising:
effecting the chemical reaction in the apparatus of claim 6.

17. A process for producing a product by a chemical reaction, comprising:
effecting the chemical reaction in the apparatus of claim 9.

18. A product comprising:
a framework, said framework forming a plurality of flow channels with adjacent flow channels having at least one common wall, and a bed of particles in at least a portion of the flow channels, wherein a bed in a channel has a cross-section of at least one and no more than twenty particles.

19. The product of claim 18 wherein the particles are catalyst particles.

20. The product of claim 19 wherein the bed cross-section in a channel is from 1-10 catalyst units.
